(19) United States
(12) Patent Application Publication
(10) Pub. No.: US 2008/0306461 A1
(43) Pub. Date: Dec. 11, 2008

(54) ALARM DEVICE FOR DIAPER

(76) Inventor: Yih-Song JAN, Taipei City (TW)

Correspondence Address: BROWDY AND NEIMARK, P.L.L.C.;624 NINTH STREET, NW, SUITE 300, WASHINGTON, DC, 20001-5303, US

(21) Appl. No.: 11/831,246

(22) Filed: Jul. 31, 2007

(30) Foreign Application Priority Data
Jun. 11, 2007 (TW) 96121039

(51) Int. Cl. A61F 13/42

(52) U.S. Cl. 604/361

(57) ABSTRACT

An alarm device for a diaper includes a substrate, a cathode partially covering a surface of the substrate, an anode partially covering the surface of the substrate and spaced from the cathode, an absorbent layer covering the surface of the substrate and connecting the cathode and the anode, and an alarm electrically connecting with the cathode and the anode. The absorbent layer is made of polymer material containing electrolyte. Once the absorbent layer absorbs water, the ions of the electrolyte in the absorbent layer can move so that a battery is formed with the cathode and the anode to turn on the alarm. By means of the aforesaid arrangement, the alarm device of the present invention sends out warning while the diaper is wetted by urine or water. There is no battery-exhausting problem so that the alarm device is convenience in use.

### BACKGROUND OF THE INVENTION

1. Field of the Invention

The present invention relates to a diaper and more particularly, to an alarm device for a diaper capable of sending out warning by light, noise or transmitting wireless signals while the diaper is wetted by urine or water.

2. Description of the Related Art

There is no alarm device in conventional diaper. The caretaker have to observe user's condition or smell the odor, even open the diaper and check, to judge whether the diaper should be changed. That is very inconvenient.

For solving this problem, a diaper with battery and alarm is presented to the public. When the diaper is wetted, a sensor in the diaper becomes short circuit, allowing the battery drives the alarm to remind the caretaker to change diaper. However, the battery and the alarm are independent from the diaper. They have to be taken off from an old diaper and then put on a new one. It is still inconvenient to the user. Besides, the battery has a limited lifetime and needs to be changed periodically. It made more troubles to the user.

### SUMMARY OF THE INVENTION

It is the primary objective of the present invention to provide an alarm device for a diaper, which installs directly on a diaper and is convenient to the user.

It is another objective of the present invention to provide an alarm device for a diaper without battery-exhausting problem.

It is another objective of the present invention to provide an alarm device for a diaper, which is capable of sending out warning while the diaper is wetted by urine or water.

To achieve the above-mentioned objectives of the present invention, the alarm device for a diaper provided by the present invention comprises a substrate, a cathode partially covering a surface of the substrate, an anode partially covering the surface of the substrate and spaced from the cathode, an absorbent layer covering the surface of the substrate and connecting the cathode and the anode, and an alarm electrically connecting with the cathode and the anode. The absorbent layer is made of polymer material containing electrolyte. Once the absorbent layer absorbs water, the ions of the electrolyte in the absorbent layer can move so that a battery is formed with the cathode and the anode to turn on the alarm.

### BRIEF DESCRIPTION OF THE DRAWINGS

FIG. 1 is a perspective view of a diaper provided with the alarm device according to a first embodiment of the present invention.

FIG. 2 is a schematic view of the alarm device according to the first embodiment of the present invention.

FIG. 3 is a sectional view along line 3-3 of FIG. 2.

FIG. 4 is a sectional view of the alarm device according to a second embodiment of the present invention.

FIG. 5 is a schematic view of the alarm device according to a third embodiment of the present invention.

### DETAILED DESCRIPTION OF THE INVENTION

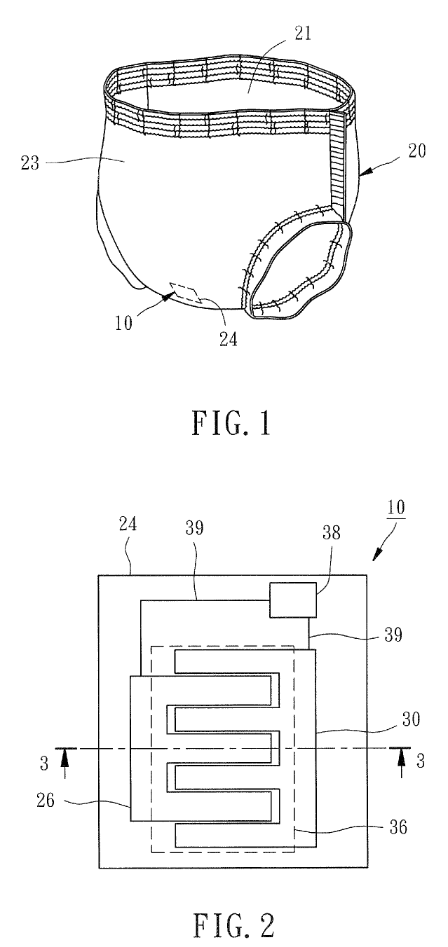

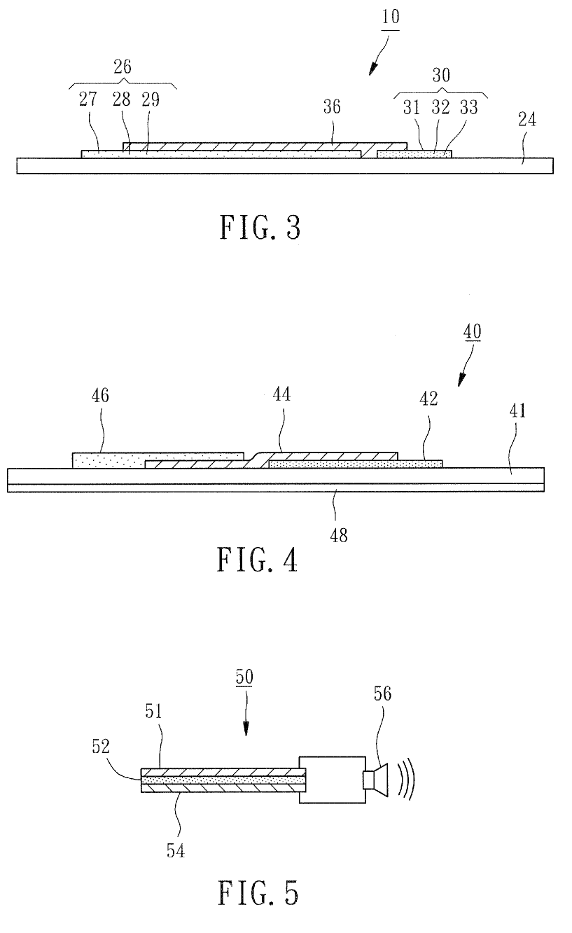

As shown in FIGS. 1-3, the alarm device 10 in accordance with the first embodiment of the present invention comprises a substrate 24, a cathode 26, an anode 30, an absorbent layer 36 and an alarm 38. The alarm device 10 is mounted on a diaper 20.

The diaper 20 is provided with a permeable surface layer 21, an absorbent inner layer (not shown) and an impervious bottom layer 23. The inner layer is capable of absorbing excreta, such as urine, excrement or sweat.

The substrate 24 is mounted in the inner layer of the diaper 20 and is located at the position that is easy to be wet by urine. The substrate 24 is made of flexible material, such as paper, carbon fiber fabric, polyethylene terephthalate or other plastic, to avoid discomforting wearer.

The cathode 26 partially covers a top surface of the substrate 24 and is shaped like a comb. The cathode 26 comprises a polymer layer 27, electricity-conducting carbon powder 28 distributed evenly in the polymer layer 27, and MnO₂ powder 29 distributed evenly in the polymer layer 27. In practice, electricity-conducting carbon powder 28 and MnO₂ powder 29 are added into liquid state polymer material and mix together, and then apply on the surface of the substrate 24 to form the cathode 26 after solidification. The MnO₂ powder 29 may be replaced by carbon (C) powder, silver oxide (Ag₂O) powder, silver peroxide (AgO) powder or a mixture thereof. It is also possible to form the cathode 26 by physical vapor deposition, chemical vapor deposition or electroplating of MnO₂, C, Ag₂O or AgO directly on the surface of the substrate 24.

The anode 30, which is shaped like a comb, partially covers the surface of the substrate 24 and spaced from the cathode 26. A gap is defined between the cathode 26 and the anode 30. The anode 30 comprises a polymer layer 31, electricity-conducting carbon powder 32 distributed evenly in the polymer layer 31, and zinc powder 33 distributed evenly in the polymer layer 31. The manufacture process is similar to the one for the cathode 26. The zinc powder may be replaced by zinc alloy powder. It is also possible to form the anode 30 by physical vapor deposition, chemical vapor deposition or electroplating of Zn or Zn alloy directly on the surface of the substrate 24.

The absorbent layer 36 covers the surface of the substrate 24 and connects the cathode 26 and the anode 30. The absorbent layer 36 is made of polymer material containing electrolyte. The ions of the electrolyte are fixed when the polymer material is dry. The cathode 26 is insulated from the anode 30 at this time. Once the polymer material absorbs water, the ions of the electrolyte can move freely so that a battery is formed with the cathode 26 and the anode 30. In practice, the electrolyte may adopt sodium hydroxide (NaOH), potassium hydroxide (KOH), lithium hydroxide (LiOH), magnesium hydroxide (Mg(OH)₂) or other salt containing OH⁻. The polymer material of the absorbent layer 36 may adopt polyvinyl alcohol (PVA), polyvinyl acetate (PVAc), polyacrylate resin, sodium polyacrylate, salt polyacrylate, charged polysaccharides or a mixture thereof, which can also be adopted by the polymer layer of the cathode 26 and the anode 30.

The alarm 38 is mounted on the substrate 24 and electrically connects with the cathode 26 and the anode 30 respectively through two wires 39. The alarm 38 is a wireless signal transmitter, such as Bluetooth of RF transmitter, capable of transmitting signals for receiving by remote receiver.

The ions of the electrolyte in the absorbent layer 36 are fixed when the diaper 20 is dry, therefore the battery-exhausting problem of the prior art does not exist. Once the inner layer of the diaper 20 absorbs urine or water to make the absorbent layer 36 wet, the ions of the electrolyte in the absorbent layer 36 can move freely so that an electric potential is formed between the cathode 26 and the anode 30, i.e. a battery is then formed. The alarm 38 can be turned on by the battery to transmit wireless signals to the remote receiver (not shown) to remind caretaker to change diaper.

It should be emphasized that the cathode 26 and the anode 30 are insulated from each other when the absorbent layer 36 is dry, therefore the user does not need to worry about whether the battery power is exhausted or not. The alarm device 10 of the present invention is mounted inside the diaper 20, thus there is no need to reinstall the battery and the alarm onto the diaper while user changes the diaper. Besides, since the absorbent layer 36 contains electrolyte, the ions of the electrolyte can move when the absorbent layer 36 is wetted not only by excreta but also by pure water without electrolyte. Therefore, the application of the alarm device of the present invention is more general. For example, the battery function is actuated even when the diaper 20 is wetted by tap water, juice or milk. In other words, the alarm device of the present invention is not only convenient in use but also general in application.

The position of the alarm device 10 can be altered according to user's requirement. For example, it may be mounted between the inner layer and the surface layer 21 of the diaper 20, or it is mounted on the surface layer 21 of the diaper 20. The alarm device 10 may be presented as an independent product. The users may buy the alarm device 10 and adhere it to their own diaper.

According to the spirit of the present invention, the shape and the structure of the cathode and the anode can be altered. As shown in FIG. 4, the alarm device 40 in accordance with the second preferred embodiment of the present invention comprises a substrate 41 covered in turn with a cathode 42, an absorbent layer 44 and an anode 46 such that the absorbent layer 44 is sandwiched between the cathode 42 and the anode 46, which is different from the structure of the first embodiment. The bottom surface of the substrate 41 is provided with an adhering layer 48 such that user may stick the alarm device 40 at proper position of the diaper.

As shown in FIG. 5, the alarm device 50 in accordance with the third preferred embodiment of the present invention has a sandwiched structure formed by a cathode 51, an absorbent layer 52 and an anode 54. The cathode 51 is made by adding electricity-conducting carbon powder and Ag₂O powder into liquid state polymer material and then forming an electrode plate by pressing. The anode 54 is made of zinc. The substrate of the aforesaid two embodiments is eliminated since both of the cathode and the anode have enough structure strength. Besides, the alarm device 50 is provided with an alarm 56 connecting with the cathode 51 and the anode 54. The alarm 56 may adopt light-emitting alarm or sound-emitting alarm for sending out warning to remind caretaker beside the wearer.

In practice, the cathode may be made by adding electricity-conducting carbon powder and Ag₂O powder into liquid state polymer material, applying them onto an auxiliary board, and then removing the auxiliary board after the polymer material consolidated. Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. An alarm device for a diaper comprising:
a substrate;
a cathode partially covering a surface of said substrate;
an anode partially covering the surface of said substrate and spaced from said cathode;
an absorbent layer covering the surface of said substrate and connecting said cathode and said anode; wherein said absorbent layer is made of polymer material containing electrolyte; and
an alarm electrically connecting with said cathode and said anode.

2. The alarm device as claimed in claim 1, wherein said cathode is made of material selected from a group consisting of manganese dioxide (MnO₂), carbon (C), silver oxide (Ag₂O), silver peroxide (AgO) and a mixture thereof.

3. The alarm device as claimed in claim 1, wherein said cathode comprises a polymer layer, electricity-conducting carbon powder distributed in said polymer layer, and powder distributed in said polymer layer and made of material selected from a group consisting of manganese dioxide (MnO₂), carbon (C), silver oxide (Ag₂O), silver peroxide (AgO) and a mixture thereof.

4. The alarm device as claimed in claim 1, wherein said anode is made of material selected from a group consisting of zinc (Zn) and zinc alloy.

5. The alarm device as claimed in claim 1, wherein said anode comprises a polymer layer, electricity-conducting carbon powder distributed in said polymer layer, and powder distributed in said polymer layer and made of material selected from a group consisting of zinc (Zn) and zinc alloy.

6. The alarm device as claimed in claim 1, wherein said electrolyte of said absorbent layer is selected from a group consisting of sodium hydroxide (NaOH), potassium hydroxide (KOH), lithium hydroxide (LiOH), magnesium hydroxide (Mg(OH)₂) and a mixture thereof.

7. The alarm device as claimed in claim 1, wherein said polymer material of said absorbent layer is selected from a group consisting of polyvinyl alcohol (PVA), polyvinyl acetate (PVAc), polyacrylate resin, sodium polyacrylate, salt polyacrylate, charged polysaccharides and a mixture thereof.

8. The alarm device as claimed in claim 1, wherein said alarm is selected from a group consisting of light-emitting alarm, sound-emitting alarm and wireless signal transmitter.

9. The alarm device as claimed in claim 1, further comprising an adhesive layer provided on a bottom surface of said substrate.

10. A diaper comprising the alarm device of claim 1.

11. An alarm device for a diaper comprising:
a cathode;
an anode;
an absorbent layer provided between said cathode and said anode; wherein said absorbent layer is made of polymer material containing electrolyte; and
an alarm electrically connecting with said cathode and said anode.

12. The alarm device as claimed in claim 11, wherein said cathode is made of material selected from a group consisting of MnO₂, C, Ag₂O, AgO and a mixture thereof.

13. The alarm device as claimed in claim 11, wherein said cathode comprises a polymer layer, electricity-conducting carbon powder distributed in said polymer layer, and powder distributed in said polymer layer and made of material selected from a group consisting of MnO₂, C, Ag₂O, AgO and a mixture thereof.

14. The alarm device as claimed in claim 11, wherein said anode is made of material selected from a group consisting of Zn and zinc alloy.

15. The alarm device as claimed in claim 11, wherein said anode comprises a polymer layer, electricity-conducting carbon powder distributed in said polymer layer, and powder distributed in said polymer layer and made of material selected from a group consisting of Zn and zinc alloy.

16. The alarm device as claimed in claim 11, wherein said electrolyte of said absorbent layer is selected from a group consisting of NaOH, KOH, LiOH, Mg(OH)₂ and a mixture thereof.

17. The alarm device as claimed in claim 11, wherein said polymer material of said absorbent layer is selected from a group consisting of polyvinyl alcohol (PVA), polyvinyl acetate (PVAc), polyacrylate resin, sodium polyacrylate, salt polyacrylate, charged polysaccharides and a mixture thereof.

18. The alarm device as claimed in claim 11, wherein said alarm is selected from a group consisting of light-emitting alarm, sound-emitting alarm and wireless signal transmitter.

19. A diaper comprising the alarm device of claim 11.
